(19) United States
(12) Patent Application Publication
(10) Pub. No.: US 2017/0216372 A1
(43) Pub. Date: Aug. 3, 2017

(54) COMPOSITIONS AND METHODS FOR FECAL MICROBIOTA-RELATED THERAPY

(71) Applicant: Crestovo LLC, Cambridge, MA (US)

(72) Inventor: Thomas Julius BORODY, Five Dock (AU)

(73) Assignee: Crestovo LLC, Cambridge, MA (US)

(21) Appl. No.: 15/486,262

(22) Filed: Apr. 12, 2017

Related U.S. Application Data
(63) Continuation of application No. PCT/US16/58938, filed on Oct. 26, 2016.
(60) Provisional application No. 62/395,761, filed on Sep. 16, 2016.
(60) Provisional application No. 62/270,159, filed on Dec. 21, 2015.
(60) Provisional application No. 62/246,441, filed on Oct. 26, 2015.

(51) Int. Cl. A61K 35/741; A61K 9/00

(52) U.S. Cl. CPC A61K 35/741; A61K 9/0053; A61K 2035/115

(57) ABSTRACT

The present application provides microbiota-related compositions, methods, and dosing regimens for treating various microbiota-related disorders with improved cure rates by replacing or supplementing or modifying a subject's colon microbiota.

### FIELD

The present disclosure relates to pharmaceutical compositions and methods suitable for the treatment of diseases in mammals. More specifically, the disclosure relates to treating various diseases, such as gastrointestinal diseases in humans using fecal microbiota-related therapy.

### BACKGROUND

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and results in increased susceptibility to pathogens, altered metabolic profiles, or induction of proinflammatory signals that can lead to local or systemic inflammation or autoimmunity. The intestinal microbiota plays a significant role in the pathogenesis of many disorders such as pathogenic infections of the gut.

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE), and other conditions such as Irritable Bowel Syndrome, Colitis, and Autism Spectrum Disorder (ASD). It entails infusions through a colonoscope, an enema or via a nasojejunal tube of human microbiota either in the form of homogenised stool, or cultured stool components such as Clostridia, to implant in the colon and thereby displace or eradicate pathogenic bacteria, e.g., C. difficile.

FMT generally has a decent success rate for treating CDI. For example, in treating CDI, FMT has been reported to achieve as high as a 90% cure rate from a single infusion. However, this means the remaining 10% CDI patients, most of whom are in the severe CDI category, still experience further relapses and may face serious life challenges or even death. Therefore, there is a need for improving FMT methods including, for example, novel and more efficacious dosing regimens to improve cure rates of various disorders treated by fecal bacteriotherapy. There is also a need in the art for rationalized, optimized, and/or individualized dosing regimens for delivery of fecal bacteriotherapy.

Delivery of FMT by upper route, including gastroscopy, nasogastric/nasojejunal tube, and lower route, including retention enema, sigmoidoscopy, or colonoscopy have all been proposed. However, endoscopic delivery requires significant health care utilization and associated cost. Therefore, there is a need to develop novel methods for FMT to be infused by a noninvasive modality. The instant application provides, inter alia, novel bacteriotherapy-based methods and regimens which would significantly reduce patient discomfort, procedure related risks, and health care costs, while offering higher efficacy compare to colonoscopic delivery. The instant application also provides, inter alia, novel dosing regimens that achieve higher response, cure, or remission rates relative to a conventional single-dosing treatment.

### SUMMARY

In one aspect, the present disclosure provides a method for treating a disorder in a subject in need thereof, the method comprising administering to the subject a pharmaceutically active dose of a therapeutic composition comprising live non-pathogenic bacteria, the dose being administered at a first dosing schedule of at least once daily or weekly for at least three consecutive days or weeks.

In one aspect, the present disclosure provides a method for treating a disorder in a subject in need thereof, the method comprising administering to the subject a pharmaceutically active dose of a therapeutic composition comprising live non-pathogenic bacteria, the dose being administered at a first dosing schedule of at least twice daily or at least twice weekly for at least two consecutive days or weeks.

In one aspect, the present disclosure provides a method for treating a disorder in a subject in need thereof, the method comprising administering to the subject a pharmaceutically active dose of a therapeutic composition comprising live non-pathogenic bacteria, the dose being administered at a first dosing schedule of at least three times daily or at least three times weekly for at least two consecutive days or weeks.

In one aspect, a first dosing schedule of a method is an initial treatment dose followed by a second dosing schedule. In another aspect, a second dosing schedule comprises a maintenance dose lower than or equal to a pharmaceutically active dose of a first dosing schedule.

In another aspect, this disclosure further provides use of any of a disclosed composition in the manufacture of a medication for the treatment of any of the disorders mentioned herein.

### DETAILED DESCRIPTION

Unless defined otherwise herein, terms are to be understood according to conventional usage by those of ordinary skill in the relevant art.

Effectiveness of a medical treatment depends on multiple variables such as the pharmaceutical composition used, its route of administration, the amount of composition administered, and the dosing schedule. This application relates to an unexpected and surprising combination of a novel dosing schedule and a low dosage which leads to a higher therapeutic efficacy.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” refers to an amount of a composition which is effective in treating the named disease, disorder or condition.

As used herein, an “intermittent dosing schedule” means that that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, an “continuous dosing schedule” refers to a dosing schedule where a therapeutic composition is administered during a treatment period without a rest period. Throughout the treatment period of a continuous dosing schedule, a therapeutic composition can be administered, for example, daily, weekly, or every other day, or every third day. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time, for example, once per day, once a week, or once in two weeks.

As used herein, “dosing interval” refers to the amount of time that elapses between multiple doses being administered to a subject.

As used herein, “primary C. difficile infection (CDI)” refers to a first or initial episode of C. difficile-associated diarrhea. Assays for detecting C. difficile in stools include, for example, stool culture, glutamate dehydrogenase enzyme immunoassay (EIA), real-time polymerase chain reaction (PCR) assay, stool cytotoxin test, EIA for detecting toxins A and B, and latex agglutination technique. These assays are well known in the art.

As used herein, “recurrent C. difficile infection (CDI)” refers to a form of CDI exhibiting one or more recurrence of C. difficile-associated diarrhea. Recurrence can be due to relapse or reinfection. An infection due to the same strain of C. difficile which caused the first episode is a relapse, while an infection with a different strain of the organism from the first episode is a reinfection.

As used herein, “microbiota,” and “flora” refer to a community of microbes that live in or on a subject's body, both sustainably and transiently, including eukaryotes, archaea, bacteria, and viruses (including bacterial viruses (i.e., phage)).

As used herein, “colony forming units” (cfu) refers to an estimate of the number of viable microorganism cells in a given sample.

As used herein, “viable” means possessing the ability to multiply.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula

$$H = - {\sum }_{i = 1}^{R} {p}_{i } ln {p}_{i}$$

where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

The present disclosure includes and relates to the use of a fecal microbiota, one or more microbial species therefrom, or an active fragment or component therefrom for the treatment and/or prophylaxis of various disease states related to the presence of ‘abnormal’ microflora in the GI tract. Many chronic diseases and disorders of the GI tract have chronic infection/infestation as their underlying pathological cause (e.g., CDI, irritable bowel syndrome, spastic colon, mucous colitis, collagenous colitis, ulcerative colitis, Crohn's disease, Johne's disease (paratuberculosis), microscopic colitis, idiopathic inflammatory bowel disease, antibiotic-associated colitis, idiopathic or simple constipation, diverticular disease, Acquired Immune Deficiency Syndrome (AIDS) enteropathy, and autistic spectrum disorder (ASD)).

In one aspect, the present disclosure provides a method for treating a disorder in a subject in need thereof, the method comprising administering to the subject a pharmaceutically active dose of a therapeutic composition comprising live non-pathogenic bacteria, the dose being administered at a first dosing schedule of at least once daily or weekly for at least two consecutive days or weeks. In one aspect, a pharmaceutically active dose is administered at least once daily or weekly for at least 3, 4, 5, 6, 7, 8, 9, 10, 11, 12, 13, 14, or 15 consecutive days or weeks. In another aspect, a pharmaceutically active dose is administered at least once daily or weekly for at least 1, 2, 3, 4, 5, 6, 7, 8, 9, 10, 11, or 12 consecutive weeks. In one aspect, a pharmaceutically active dose is administered at least once daily or weekly for at most 4, 5, 6, 7, 8, 9, 10, 11, 12, 13, 14, 15, 16, 17, 18, 19, or 20 consecutive days. In another aspect, a pharmaceutically active dose is administered at least once daily or weekly for at most 1, 2, 3, 4, 5, 6, 7, 8, 9, 10, 11, or 12 consecutive days or weeks. In a further aspect, a pharmaceutically active dose is administered at least once for at least 1, 2, 3, 4, 5, 6, 7, 8, 9, 10, 11, or 12 consecutive years, chronically for a subject's entire life span, or an indefinite period of time.

In one aspect, the present disclosure provides a method for treating a disorder in a subject in need thereof, the method comprising administering to the subject a pharmaceutically active dose of a therapeutic composition comprising live non-pathogenic bacteria, the dose being administered at a first dosing schedule of at least twice daily or at least twice weekly for at least two consecutive days or weeks. In one aspect, a pharmaceutically active dose is administered at least twice daily or at least twice weekly for at least 3, 4, 5, 6, 7, 8, 9, 10, 11, 12, 13, 14, or 15 consecutive days or weeks. In another aspect, a pharmaceutically active dose is administered at least twice daily or at least twice weekly for at least 1, 2, 3, 4, 5, 6, 7, 8, 9, 10, 11, or 12 consecutive weeks. In one aspect, a pharmaceutically active dose is administered at least twice daily or at least twice weekly for at most 4, 5, 6, 7, 8, 9, 10, 11, 12, 13, 14, 15, 16, 17, 18, 19, or 20 consecutive days or weeks. In another aspect, a pharmaceutically active dose is administered at least twice daily or at least twice weekly for at most 1, 2, 3, 4, 5, 6, 7, 8, 9, 10, 11, or 12 consecutive weeks. In a further aspect, a pharmaceutically active dose is administered at least twice for at least 1, 2, 3, 4, 5, 6, 7, 8, 9, 10, 11, or 12 consecutive years, chronically for a subject's entire life span, or an indefinite period of time.

In one aspect, the present disclosure provides a method for treating a disorder in a subject in need thereof, the method comprising administering to the subject a pharmaceutically active dose of a therapeutic composition comprising live non-pathogenic bacteria, the dose being administered at a first dosing schedule of at least three times daily or at least three times weekly for at least two consecutive days or weeks. In one aspect, a pharmaceutically active dose is administered at least three times daily or at least three times weekly for at least 3, 4, 5, 6, 7, 8, 9, 10, 11, 12, 13, 14, or 15 consecutive days or weeks. In another aspect, a pharmaceutically active dose is administered at least three times daily or at least three times weekly for at least 1, 2, 3, 4, 5, 6, 7, 8, 9, 10, 11, or 12 consecutive weeks. In one aspect, a pharmaceutically active dose is administered at least three times daily or at least three times weekly for at most 4, 5, 6, 7, 8, 9, 10, 11, 12, 13, 14, 15, 16, 17, 18, 19, or 20 consecutive days or weeks. In another aspect, a pharmaceutically active dose is administered at least three times daily or at least three times weekly for at most 1, 2, 3, 4, 5, 6, 7, 8, 9, 10, 11, or 12 consecutive weeks. In a further aspect, a pharmaceutically active dose is administered at least three times for at least 1, 2, 3, 4, 5, 6, 7, 8, 9, 10, 11, or 12 consecutive years, chronically for a subject's entire life span, or an indefinite period of time.

In one aspect, the present disclosure provides a method of treating a disorder having an underlying gastrointestinal condition in a subject in need thereof, where the method comprises administering orally to the subject a pharmaceutically active dose of a composition comprising live, non-pathogenic, synthetic bacterial mixture or live, non-pathogenic, purified or extracted, fecal microbiota, where the dose is administered at a dosing schedule of at least twice daily or at least twice weekly for at least three consecutive days or weeks.

In one aspect, a first dosing schedule of a method is followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, a subject being treated is a subject already with a disease. In another aspect, a subject being treated is a subject in which a disease is to be prevented. In another aspect, a subject being treated is predisposed or susceptible to a disease. In another aspect, a subject being treated is a subject diagnosed as having a disease. In one aspect, a subject being treated is a patient in need thereof.

In one aspect, a subject being treated is a human patient. In one aspect, a patient is a male patient. In one aspect, a patient is a female patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old.

In one aspect, a method comprises administering a therapeutic composition orally, by enema, or via rectal suppository. In one aspect, a therapeutic composition administered herein is formulated as an enteric coated capsule or an enteric coated microcapsule, or formulated as part of or administered together with a food, a food additive, a dairy-based product, a soy-based product or a derivative thereof, a jelly, or a yogurt. In another aspect, a therapeutic composition administered herein is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated microcapsules containing a therapeutic composition.

In an aspect, a therapeutic composition comprises a liquid culture. In another aspect, a therapeutic composition is lyophilized, pulverized and powdered. It may then be infused, dissolved such as in saline, as an enema. Alternatively the powder may be encapsulated as enteric-coated capsules for oral administration. These capsules may take the form of enteric-coated microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition administered herein is in a liquid, frozen, freeze-dried, spray-dried, lyophilized, or powder form. In a further aspect, a therapeutic composition administered herein is formulated as a delayed or gradual enteric release form. In another aspect, a therapeutic composition administered herein comprises an excipient, a saline, a buffer, a buffering agent, or a fluid-glucose-cellobiose agar (RGCA) media. In another aspect, a therapeutic composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a therapeutic composition administered herein further comprises an acid suppressant, an antacid, an H2 antagonist, a proton pump inhibitor or a combination thereof. In one aspect, a therapeutic composition administered herein substantially free of non-living matter. In another aspect, a therapeutic composition administered herein substantially free of acellular material selected from the group consisting of residual fiber, DNA, viral coat material, and non-viable material.

In one aspect, a method further comprises pretreating a subject with an antibiotic composition prior to administering a therapeutic composition. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.

In one aspect, a method is for treating a disorder selected from the group consisting of primary Clostridium difficile infection and recurrent C. difficile infection. In another aspect, a method is for treating a disorder selected from the group consisting of Crohn's disease, primary C. difficile infection, recurrent C. difficile infection, autism spectrum disorder (ASD), constipation predominant functional bowel disease (FBD), pain predominant FBD, upper abdominal FBD, non-ulcer dyspepsia (NUD), gastro-esophageal reflux, indeterminate colitis, microscopic colitis, pseudomembranous colitis, viral gastroenteritis, Norwalk viral gastroenteritis, rotavirus gastroenteritis, AIDS related gastroenteritis, non-rheumatoid factor positive arthritis, Lyme disease, systemic lupus, idiopathic thrombocytopenic purpura, Sjogren's syndrome, hemolytic uremic syndrome or scleroderma, Guillain-Barré syndrome, Chronic Inflammatory Demyelinating Polyneuropathy, chronic depression, schizophrenia, psychotic disorders, manic depressive illness, Asperger syndrome, Rett syndrome, attention deficit hyperactivity disorder (ADHD), attention deficit disorder (ADD), sudden infant death syndrome (SIDS), anorexia nervosa, acne, halitosis, collagenous colitis, indeterminate colitis, cyclic vomiting, relapsing diverticulitis, rheumatoid arthritis, sacroileitis, chronic nausea, ulcerative colitis with sclerosing cholangitis, Parkinson's disease, Shigella infection, Vancomycin Resistant Enterococci (VRE) infection, Methicillin Resistant Staphylococcus Aureus (MRSA) infection, and carbapenem-resistant Klebsiella pneumoniae. In another aspect, a method is for treating a disorder which exhibits or is associated with gastrointestinal dysbiosis. In another aspect, a method increase bacterial diversity in said subject's gastrointestinal tract.

In one aspect, a therapeutic composition or method can be used to treat chronic infections in a wide range of chronic disorders such as Crohn's colitis, irritable bowel syndrome (such as IBS-D, IBS-C, and IBS-M), particularly when characterized by chronic abdominal pain, bloating, or excessive flatulence, together with chronic diarrhea or alternating constipation/diarrhea, and also in spastic colon, mucous colitis, collagenous colitis, ulcerative colitis, microscopic colitis, idiopathic inflammatory bowel disease, antibiotic-associated colitis, idiopathic or simple constipation, diverticular disease and AIDS enteropathy. In another aspect, a therapeutic composition or method can be used to treat other gastrointestinal disorders of unexplained etiology such as polyposis coli and colonic polyps, which may be influenced by the local bowel microflora.

In another aspect, a method or a dosing regimen can be used to treat a chronic gastrointestinal infection with a specific microorganism such as Clostridium difficile, Mycobacterium aviun paratuberculosis, Shigella sp., Yersinia sp., Campylobacter sp., Aeromonas sp., Escherichia coli, Cryptosporidium sp., Amoebae, Blastocystitis hominis, and Giardia, and a chronic viral infection, and of small bowel bacterial overgrowth.

In one aspect, a method or a dosing regimen can be used to treat a liver disease, migraines, chronic fatigue syndrome, and other neurological syndromes such as, multiple sclerosis, amyotrophic lateral sclerosis, myasthenia gravis, Parkinson's disease, Alzheimer's disease, Chronic Inflammatory Demyelinating Polyneuropathy (CIDP), Guillain-Barré Syndrome, and other degenerative disorders.

In another aspect, a method or a dosing regimen can be used to treat a joint disease, such as rheumatoid arthritis, the non-rheumatoid arthritidies including, ankylosing spondylitis, and Reiter's syndrome. In a further aspect, a method or a dosing regimen can be used to treat a syndrome with an immune mediated component such as glomerulonephritis, hemolytic uremic syndrome, juvenile diabetes mellitus, Behçet's syndrome, coeliac disease and dermatitis herpetiformis. Similarly, syndromes with an immune complex mediated component, such as scleroderma, systemic lupus erythematosus, mixed cryoglobulinaemia, polyarteritis, familial Mediterranean fever, amyloidosis, and the various presentations of such syndromes, together with such “idiopathic” states as chronic urticaria, may be manifestations of variations of immune regulated responses to related bowel-origin pathogens chronically shedding their antigen(s), toxins or biological response modifiers into the circulation. Other chronic conditions such as acne, and chronic idiopathic pseudo-obstructive syndrome, may well be influenced by similar mechanisms. In one aspect, a therapeutic composition, dosing regimen, or method can be used to treat any of these diseases or disorders.

In one aspect, the present disclosure provides a method for the treatment and/or prophylaxis of a chronic disorder associated with the presence in the gastrointestinal tract of a mammalian host of abnormal or an abnormal distribution of microflora, which method comprises administering an effective amount of a therapeutic composition following a dosing schedule provided herein. Such a disorder includes but is not limited to those conditions in the following categories: gastro-intestinal disorders including irritable bowel syndrome or spastic colon, functional bowel disease (FBD), including constipation predominant FBD, pain predominant FBD, upper abdominal FBD, non-ulcer dyspepsia (NUD), gastro-esophageal reflux, inflammatory bowel disease including Crohn's disease, ulcerative colitis, indeterminate colitis, collagenous colitis, microscopic colitis, chronic Clostridium difficile infection, pseudomembranous colitis, mucous colitis, antibiotic associated colitis, idiopathic or simple constipation, diverticular disease, AIDS enteropathy, small bowel bacterial overgrowth, coeliac disease, polyposis coli, colonic polyps, chronic idiopathic pseudo obstructive syndrome; chronic gut infections with specific pathogens including bacteria, viruses, fungi and protozoa; viral gastrointestinal disorders, including viral gastroenteritis, Norwalk viral gastroenteritis, rotavirus gastroenteritis, AIDS related gastroenteritis; liver disorders such as primary biliary cirrhosis, primary sclerosing cholangitis, fatty liver or cryptogenic cirrhosis; rheumatic disorders such as rheumatoid arthritis, non-rheumatoid arthritidies, non-rheumatoid factor positive arthritis, ankylosing spondylitis, Lyme disease, and Reiter's syndrome; immune mediated disorders such as glomerulonephritis, hemolytic uremic syndrome, juvenile diabetes mellitus, mixed cryoglobulinaemia, polyarteritis, familial Mediterranean fever, amyloidosis, scleroderma, systemic lupus erythematosus, and Behçet's syndrome; autoimmune disorders including systemic lupus, idiopathic thrombocytopenic purpura, Sjogren's syndrome, hemolytic uremic syndrome or scleroderma; neurological syndromes such as chronic fatigue syndrome, migraine, multiple sclerosis, amyotrophic lateral sclerosis, myasthenia gravis, Guillain-Barré syndrome, Parkinson's disease, Alzheimer's disease, Chronic Inflammatory Demyelinating Polyneuropathy, and other degenerative disorders; psychiatric disorders including chronic depression, schizophrenia, psychotic disorders, manic depressive illness; regressive disorders including Asperger syndrome, Rett syndrome, attention deficit hyperactivity disorder (ADHD), and attention deficit disorder (ADD); the regressive disorder, autism; sudden infant death syndrome (SIDS), anorexia nervosa; dermatological conditions such as, chronic urticaria, acne, dermatitis herpetiformis and vasculitic disorders. In another aspect, the present disclosure also provides a therapeutic composition for use in a method of treating any one of the diseases mentioned in this paragraph. In an aspect, the present disclosure also discloses the use of a fecal microbiota in the manufacture of a medicament for the treatment of any one of the diseases mentioned in this paragraph, where the medicament is prepared for administration with another non-fecal bacterium. In another aspect, the present disclosure also discloses the use of another non-fecal bacterium in the manufacture of a medicament for the treatment of any one of the diseases mentioned in this paragraph, where the medicament is prepared for administration with a fecal microbiota. In a further aspect, the present disclosure also includes a product containing another non-fecal bacterium and a fecal microbiota as a combined preparation for simultaneous, separate, or sequential use in the treatment of any one of the diseases mentioned in this paragraph.

In one aspect, a method achieves a remission, cure, response, or resolution rate of a disorder of at least about 30%, 40%, 50%, 60%, 70%, 80%, 85%, 90%, 95%, 97%, or 99%. In one aspect, a first dosing schedule of a method achieves a higher remission, cure, response, or resolution rate of a disorder compared to a dosing schedule of a single dose of the same composition. In one aspect, a first dosing schedule achieves at least about 2%, 3%, 4%, 5%, 6%, 7%, 8%, 10%, 12%, 14%, 16%, 18%, 20%, 25%, 30%, 35%, 40%, 45%, 50%, 55%, 60%, 65%, 70%, 75%, 80%, 85%, or 90% higher remission, cure, response, or resolution rate compared to a single-dosing schedule. In another aspect, a first dosing schedule of a method achieves a higher remission, cure, response, or resolution rate of the disorder compared to a single dosing schedule of the composition, where the total amount of viable non-pathogenic bacteria administered are substantially similar between the first dosing schedule and the single dosing schedule. In one aspect, a first dosing schedule achieves at least about 2%, 3%, 4%, 5%, 6%, 7%, 8%, 10%, 12%, 14%, 16%, 18%, 20%, 25%, 30%, 35%, 40%, 45%, 50%, 55%, 60%, 65%, 70%, 75%, 80%, 85%, or 90% higher remission, cure, response, or resolution rate compared to a single-dosing schedule, where the total amount of viable non-pathogenic bacteria administered are substantially similar between the first dosing schedule and the single dosing schedule.

In one aspect, a pharmaceutically active dose comprises at least about 10⁵, 10⁶, 10⁷, 10⁸, 10⁹, 10¹⁰, 10¹¹, 10¹², or 10¹³ cfu. In another aspect, a pharmaceutically active dose comprises at most about 10⁵, 10⁶, 10⁷, 10⁸, 10⁹, 10¹⁰, 10¹¹, 10¹², or 10¹³ cfu. In a further aspect, a pharmacologically active dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², or 10¹³ cells or spores. In another aspect, a pharmaceutically active or therapeutic effective dose comprises at most about 10⁵, 10⁶, 10⁷, 10⁸, 10⁹, 10¹⁰, 10¹¹, 10¹², or 10¹³ total cells or spores. In a further aspect, a pharmacologically active or therapeutic effective dose is selected from the group consisting of from 10⁴ to 10⁵, from 10⁵ to 10⁶, from 10⁶ to 10⁷, from 10⁷ to 10⁸, from 10⁵ to 10⁸, from 10⁶ to 10⁸, from 10⁵ to 10⁹, from 10⁵ to 10¹⁰, from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises a total cell count of an approximate amount selected from the group consisting of 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², and 9×10¹².

In an aspect, a therapeutic composition comprises non-pathogenic spores of one or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium sub terminale, Clostridium symbiosum, Clostridium tertium, Clostridium tetani, Clostridium welchii, and Clostridium villosum.

In one aspect, a therapeutic composition administered herein comprises fecal bacteria. In one aspect, a therapeutic composition administered herein comprises one or more microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, and Monilia.

In one aspect, a therapeutic composition administered herein comprises at least one, at least two, at least three, at least four, at least five, at least six, or at least seven fecal microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, and a combination thereof.

In one aspect, a therapeutic composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a therapeutic composition administered herein comprises no viable Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Fine goldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, or a combination thereof.

In one aspect, a therapeutic composition administered herein comprises a fecal microbiota. In another aspect, the preparation of a fecal microbiota used herein involves a treatment selected from the group consisting of ethanol treatment, detergent treatment, heat treatment, irradiation, and sonication. In another aspect, the preparation of a fecal microbiota used herein involves no treatment selected from the group consisting of ethanol treatment, detergent treatment, heat treatment, irradiation, and sonication. In one aspect, the preparation of a fecal microbiota used herein involves a separation step selected from the group consisting of density gradients, filtration (e.g., sieves, nylon mesh), and chromatography. In another aspect, the preparation of a fecal microbiota used herein involves no separation step selected from the group consisting of density gradients, filtration (e.g., sieves, nylon mesh), and chromatography. In another aspect, a fecal microbiota used herein comprises a donor's entire fecal microbiota. In another aspect, a therapeutic composition administered herein comprises a fecal microbiota substantially free of eukaryotic cells from the fecal microbiota's donor.

In another aspect, a therapeutic composition administered herein comprises a fecal microbiota further supplemented, spiked, or enhanced with a fecal microorganism. In one aspect, a fecal microbiota is supplemented with a bacterium of Coprococcus, Prevotella, Veillonellaceae, Firmicutes, Gammaproteobacteria, or a combination thereof. In another aspect, a therapeutic composition administered herein comprises a fecal microbiota further supplemented with fecal bacterial spores. In one aspect, fecal bacterial spores are Clostridium spores or Bacillus spores.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless.

In an aspect, a therapeutic composition provided or administered herein comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In an aspect, a therapeutic composition provided or administered herein comprises a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided or administered herein comprises, consists of, or consists essentially of, particles of non-living material and/or particles of biological material of a fecal sample that passes through a sieve having a sieve size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.2 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human. In an aspect, a therapeutic composition provided or administered herein comprises an extract of human feces where the composition is substantially odorless. In an aspect, a therapeutic composition provided or administered herein comprises fecal material or a fecal floral preparation in a lyophilized, crude, semi-purified or purified formulation.

In an aspect, a fecal microbiota in a therapeutic composition comprises highly refined or purified fecal flora, e.g., substantially free of non-floral fecal material. In an aspect, a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a fecal microbiota in a therapeutic composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a fecal microbiota in a therapeutic composition comprises a donor's entire or full-spectrum fecal microbiota, reconstituted fecal material, or synthetic fecal material. In another aspect, the fecal microbiota in a therapeutic composition comprises no antibiotic resistant population. In another aspect, a therapeutic composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a fecal microbiota in a therapeutic composition used herein is derived from disease-screened fresh homologous feces or equivalent freeze-dried and reconstituted feces. In an aspect, a fresh homologous feces does not include an antibiotic resistant population. In another aspect, a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eubacterium, Fusobacterium, Propionibacterium, Lactobacillus, anaerobic cocci, Ruminococcus, Escherichia coli, Gemmiger, Clostridium, Desulfomonas, Peptostreptococcus, Bifidobacterium.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is in the form of: an enema composition which can be reconstituted with an appropriate diluent; enteric-coated capsules; enteric-coated microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated microcapsules of the composition, powder, jelly, or liquid.

In an aspect, a treatment method effects a cure, reduction of the symptoms, or a percentage reduction of symptoms of a disorder. The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

Disclosed methods, including these, can be applicable to animals in general, in particular humans and economically significant domestic animals, such as cattle, sheep, horses, pigs, goats etc. In one aspect, these methods can be especially useful in the treatment of the various forms of necrotizing enterocolitis which can be a major problem in animal stocks. When treating animals, the appropriate composition of microflora will vary according to the species being treated and the constituent normal flora known to inhabit the gut.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, for example, U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A therapeutic composition used herein can be flavored.

In an aspect, a composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

The following paragraphs list exemplary embodiments of the present disclosure.

### Embodiment 1

A method for treating a disorder in a subject in need thereof, the method comprising administering to the subject a pharmaceutically active dose of a composition comprising live non-pathogenic bacteria, the dose being administered at a first dosing schedule of at least twice a week.

### Embodiment 2

The method of Embodiment 1, wherein the dose is administered for at least 1, 2, 3, 4, 5, 6, 7, 8, 9, 10, 11, or 12 weeks

### Embodiment 3

A method for treating a disorder in a subject in need thereof, the method comprising administering to the subject a pharmaceutically active dose of a composition comprising live non-pathogenic bacteria, the dose being administered at a first dosing schedule of at least once daily for at least two consecutive days.

### Embodiment 4

The method of Embodiment 3, wherein the dose is administered at least once daily or weekly for at least 3, 4, 5, 6, 7, 8, 9, 10, 11, 12, 13, 14, or 15 consecutive days or weeks.

### Embodiment 5

The method of Embodiment 3, wherein the dose is administered at least once daily or weekly for at least 1, 2, 3, 4, 5, 6, 7, 8, 9, 10, 11, or 12 consecutive weeks.

### Embodiment 6

The method of Embodiment 3, wherein the dose is administered at least once daily or weekly for at most 3, 4, 5, 6, 7, 8, 9, 10, 11, 12, 13, 14, 15, 16, 17, 18, 19, or 20 consecutive days or weeks.

### Embodiment 7

The method of Embodiment 3, wherein the dose is administered at least once daily or weekly for at most 1, 2, 3, 4, 5, 6, 7, 8, 9, 10, 11, or 12 consecutive weeks.

### Embodiment 8

A method for treating a disorder in a subject in need thereof, the method comprising administering to the subject a pharmaceutically active dose of a composition comprising live non-pathogenic bacteria, the dose being administered at a first dosing schedule of at least twice daily or weekly for at least two consecutive days or weeks.

### Embodiment 9

The method of Embodiment 8, wherein the dose is administered at least twice daily or at least twice weekly for at least 3, 4, 5, 6, 7, 8, 9, 10, 11, 12, 13, 14, or 15 consecutive days or weeks.

### Embodiment 10

The method of Embodiment 8, wherein the dose is administered at least twice daily or at least twice weekly for at least 1, 2, 3, 4, 5, 6, 7, 8, 9, 10, 11, or 12 consecutive weeks.

### Embodiment 11

The method of Embodiment 8, wherein the dose is administered at least twice daily or at least twice weekly for at most 3, 4, 5, 6, 7, 8, 9, 10, 11, 12, 13, 14, 15, 16, 17, 18, 19, or 20 consecutive days or weeks.

### Embodiment 12

The method of Embodiment 8, wherein the dose is administered at least twice daily at least twice weekly for at most 1, 2, 3, 4, 5, 6, 7, 8, 9, 10, 11, or 12 consecutive weeks.

### Embodiment 13

A method for treating a disorder in a subject in need thereof, the method comprising administering to the subject a pharmaceutically active dose of a composition comprising live non-pathogenic bacteria, the dose being administered at a first dosing schedule of at least three times daily for at least one day.

### Embodiment 14

The method of Embodiment 13, wherein the dose is administered at least three times daily or at least three times weekly for at least 2, 3, 4, 5, 6, 7, 8, 9, 10, 11, 12, 13, 14, or 15 consecutive days or weeks.

### Embodiment 15

The method of Embodiment 13, wherein the dose is administered at least three times daily or at least three times weekly for at most 2, 3, 4, 5, 6, 7, 8, 9, 10, 11, 12, 13, 14, or 15 consecutive days or weeks.

### Embodiment 16

The method of any one of Embodiments 1 to 15, wherein the administering comprises administering orally, by enema, or via rectal suppository.

### Embodiment 17

The method of any one of Embodiments 1 to 15, wherein the composition is formulated as an enteric coated capsule, an acid-resistant, enteric-coated capsule, an enteric coated microcapsule, or formulated as part of a food, a food additive, a dairy-based product, a soy-based product or a derivative thereof, a jelly, or a yogurt.

### Embodiment 18

The method of any one of Embodiments 1 to 15, wherein the disorder is selected from the group consisting of primary Clostridium difficile infection and recurrent C. difficile infection.

### Embodiment 19

The method of any one of Embodiments 1 to 15, wherein the disorder is selected from the group consisting of Crohn's disease, primary C. difficile infection, recurrent C. difficile infection, autism spectrum disorder (ASD), constipation predominant functional bowel disease (FBD), pain predominant FBD, upper abdominal FBD, non-ulcer dyspepsia (NUD), gastro-esophageal reflux, indeterminate colitis, microscopic colitis, pseudomembranous colitis, viral gastroenteritis, Norwalk viral gastroenteritis, rotavirus gastroenteritis, AIDS related gastroenteritis, non-rheumatoid factor positive arthritis, Lyme disease, systemic lupus, idiopathic thrombocytopenic purpura, Sjogren's syndrome, hemolytic uremic syndrome or scleroderma, Guillain-Barré syndrome, Chronic Inflammatory Demyelinating Polyneuropathy, chronic depression, schizophrenia, psychotic disorders, manic depressive illness, Asperger syndrome, Rett syndrome, attention deficit hyperactivity disorder (ADHD), attention deficit disorder (ADD), sudden infant death syndrome (SIDS), anorexia nervosa, acne, halitosis, collagenous colitis, indeterminate colitis, cyclic vomiting, relapsing diverticulitis, rheumatoid arthritis, sacroileitis, chronic nausea, ulcerative colitis with sclerosing cholangitis, Parkinson's disease, Shigella infection, Vancomycin Resistant Enterococci (VRE) infection, Methicillin Resistant Staphylococcus Aureus (MRSA) infection, and carbapenem-resistant Klebsiella pneumoniae.

### Embodiment 20

The method of any one of Embodiments 1 to 15, wherein the disorder exhibits or is associated with gastrointestinal dysbiosis.

### Embodiment 21

The method of any one of Embodiments 1 to 15, wherein the method increase bacterial diversity in the subject's gastrointestinal tract.

### Embodiment 22

The method of any one of Embodiments 1 to 15, wherein the method achieves a remission, cure, response, or resolution rate of the disorder of at least about 30%, 40%, 50%, 60%, 70%, 80%, 85%, 90%, 95%, 97%, or 99%.

### Embodiment 23

The method of any one of Embodiments 1 to 15, wherein the first dosing schedule achieves a higher remission, cure, response, or resolution rate of the disorder compared to a dosing schedule of a single dose of the composition.

### Embodiment 24

The method of Embodiment 23, wherein the first dosing schedule achieves at least about 2%, 3%, 4%, 5%, 6%, 7%, 8%, 10%, 12%, 14%, 16%, 18%, 20%, 25%, 30%, 35%, 40%, 45%, 50%, 55%, 60%, 65%, 70%, 75%, 80%, 85%, or 90% higher remission, cure, response, or resolution rate compared to the single-dosing schedule.

### Embodiment 25

The method any one of Embodiments 1 to 15, wherein the first dosing schedule achieves a higher remission, cure, response, or resolution rate of the disorder compared to a single dosing schedule of the composition, and wherein the total amount of viable non-pathogenic bacteria administered are substantially similar between the first dosing schedule and the single dosing schedule.

### Embodiment 26

The method of Embodiment 25, wherein the first dosing schedule achieves at least about 2%, 3%, 4%, 5%, 6%, 7%, 8%, 10%, 12%, 14%, 16%, 18%, 20%, 25%, 30%, 35%, 40%, 45%, 50%, 55%, 60%, 65%, 70%, 75%, 80%, 85%, or 90% higher remission, cure, response, or resolution rate compared to the single-dosing schedule.

### Embodiment 27

The method of any one of Embodiments 1 to 15, wherein the pharmaceutically active dose comprises at least about 10⁵, 10⁶, 10⁷, 10⁸, 10⁹, 10¹⁰, 10¹¹, 10¹², or 10¹³ cfu.

### Embodiment 28

The method of any one of Embodiments 1 to 15, wherein the pharmaceutically active dose comprises at most about 10⁵, 10⁶, 10⁷, 10⁸, 10⁹, 10¹⁰, 10¹¹, 10¹², or 10¹³ cfu.

### Embodiment 29

The method of any one of Embodiments 1 to 15, wherein the pharmacologically active dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu.

### Embodiment 30

The method of any one of Embodiments 1 to 15, wherein the composition comprises non-pathogenic spores of one or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

### Embodiment 31

The method of any one of Embodiments 1 to 15, wherein the composition is in a liquid, frozen, freeze-dried, spray-dried, lyophilized, or powder form.

### Embodiment 32

The method of any one of Embodiments 1 to 15, wherein the composition is formulated as a delayed or gradual enteric release form.

### Embodiment 33

The method of any one of Embodiments 1 to 15, wherein the composition comprises an excipient, a saline, a buffer, a buffering agent, or a fluid-glucose-cellobiose agar (RGCA) media.

### Embodiment 34

The method of any one of Embodiments 1 to 15, wherein the composition comprises a cryoprotectant.

### Embodiment 35

The method of Embodiment 34, wherein the cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

### Embodiment 36

The method of any one of Embodiments 1 to 15, wherein the composition further comprises an acid suppressant, an antacid, an H2 antagonist, a proton pump inhibitor or a combination thereof.

### Embodiment 37

The method of any one of Embodiments 1 to 15, wherein the composition is substantially free of non-living matter.

### Embodiment 38

The method of any one of Embodiments 1 to 15, wherein the composition is substantially free of acellular material selected from the group consisting of residual fiber, DNA, viral coat material, and non-viable material.

### Embodiment 39

The method of any one of Embodiments 1 to 15, wherein the subject is pretreated with an antibiotic prior to administration of the composition.

### Embodiment 40

The method of Embodiment 39, wherein the antibiotic is selected from the group consisting of rifabutin, clarithromycin, clofazimine, vancomycin, rifampicin, nitroimidazole, chloramphenicol, and a combination thereof.

### Embodiment 41

The method of Embodiment 39, wherein the antibiotic is selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.

### Embodiment 42

The method of any one of Embodiments 1 to 15, wherein the first dosing schedule is followed by a second dosing schedule.

### Embodiment 43

The method of Embodiment 42, wherein the second dosing schedule comprises a maintenance dose lower or equal to the pharmaceutically active dose.

### Embodiment 44

The method of Embodiment 43, wherein the second dosing schedule lasts for at least about 2, 4, 6, 8, 10, 12, 18, 24, 36, 48, 72, or 96 months.

### Embodiment 45

The method of Embodiment 43, wherein the second dosing schedule lasts permanently.

### Embodiment 46

The method of Embodiment 42, wherein the interval between the first and second dosing schedules is at least about 1, 2, 3, 4, 5, 6, 7, 8, 9, 10, 11, or 12 weeks.

### Embodiment 47

The method of Embodiment 42, wherein the second dosing schedule is an continuous dosing schedule.

### Embodiment 48

The method of Embodiment 42, wherein the second dosing schedule is an intermittent dosing schedule.

### Embodiment 49

The method of Embodiment 48, wherein the intermittent dosing schedule comprises a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days.

### Embodiment 50

The method of any one of Embodiments 1 to 15, wherein the live non-pathogenic bacteria are fecal bacteria.

### Embodiment 51

The method of any one of Embodiments 1 to 15, wherein the composition comprises a fecal microbiota.

### Embodiment 52

The method of Embodiment 51, wherein the fecal microbiota is further supplemented with a fecal microorganism.

### Embodiment 53

The method of Embodiment 52, wherein the fecal microorganism is selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonif ractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, Desuifomonas pigra, Lactobacillus G, Succinivibrio A, and a combination thereof.

### Embodiment 54

The method of Embodiment 51, wherein the fecal microbiota is further supplemented with bacterial spores.

### Embodiment 55

The method of Embodiment 54, wherein the bacterial spores are Clostridium spores or Bacillus spores.

### Embodiment 56

The method of Embodiment 51, wherein the preparation of the fecal microbiota involves a treatment selected from the group consisting of ethanol treatment, detergent treatment, heat treatment, irradiation, and sonication.

### Embodiment 57

The method of Embodiment 51, wherein the preparation of the fecal microbiota involves no treatment selected from the group consisting of ethanol treatment, detergent treatment, heat treatment, irradiation, and sonication.

### Embodiment 58

The method of Embodiment 51, wherein the preparation of the fecal microbiota involves a separation step selected from the group consisting of density gradients, filtration, and chromatography.

### Embodiment 59

The method of Embodiment 51, wherein the preparation of the fecal microbiota involves no separation step selected from the group consisting of density gradients, filtration, and chromatography.

### Embodiment 60

The method of Embodiment 51, wherein the fecal microbiota comprises a donor's entire fecal microbiota.

### Embodiment 61

The method of Embodiment 51, wherein the composition is substantially free of eukaryotic cells from the fecal microbiota's donor.

### Embodiment 62

The method of Embodiment 51, wherein the fecal microbiota is from reconstituted fecal material.

### Embodiment 63

The method of Embodiment 51, wherein the fecal microbiota is from synthetic fecal material.

### Embodiment 64

The method of Embodiment 51, wherein the fecal microbiota comprises no antibiotic resistant population.

### Embodiment 65

The method of Embodiment 51, wherein the fecal microbiota comprises a preparation of viable flora in proportional content that resembles a normal healthy human fecal flora.

### Embodiment 66

The method of Embodiment 51, wherein the fecal microbiota comprises bacteria from at least seven different families.

### Embodiment 67

The method of Embodiment 51, wherein the fecal microbiota has a Shannon Diversity Index of 0.4-5.0.

### Embodiment 68

The method of Embodiment 51, wherein the fecal microbiota comprises one or more microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, and Monilia.

### Embodiment 69

The method of Embodiment 51, wherein the fecal microbiota comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof.

### Embodiment 70

The method of Embodiment 51, wherein the fecal microbiota comprises one or more microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, Desuifomonas pigra, Lactobacillus G, Succinivibrio A, and a combination thereof.

### Embodiment 71

The method of Embodiment 51, wherein the fecal microbiota comprises no viable Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, or a combination thereof.

### Embodiment 72

A method for treating a Clostridium difficile infection (CDI) in a subject in need thereof, said method comprising administering to said subject a pharmaceutically active dose of a composition comprising a donor's substantially complete microbiota, said dose being administered at a first dosing schedule having 2 or more days.

### Embodiment 73

The method of Embodiment 72, wherein said dose is administered over two consecutive days.

### Embodiment 74

The method of claim 72 or 73, wherein said dose is administered for at least twice a day.

### Embodiment 75

The method of Embodiment 74, wherein said first dosing schedule achieves an increase in the remission or cure rate of said CDI compared to a second dosing schedule where a similar total amount of said composition is administered in a single day.

### Embodiment 76

The method of Embodiment 75, wherein said similar total amount of said composition is administered at once in said second dosing schedule.

### Embodiment 77

The method of Embodiment 75, wherein said increase is at least about 5%, 8%, 10%, 15%, 20%, 25%, or 30%.

### Examples

### Example 1. Preparation of Fecal Microbiota

Fecal microbiota (full-spectrum microbiota) is prepared essentially according to protocols published in US2014/0147417 or WO2014/152484. Summarized below is an exemplary protocol.

Potential fecal microbiota donors are screened according to a list of criteria used to exclude unsuitable donors. Potential fecal microbiota donors are excluded if they have received antibiotics, laxatives, diet pills, immunomodulators or chemotherapy in the preceding three months. Potential fecal microbiota donors are excluded if they have a history of all known infectious diseases, morbid obesity, diabetes, irritable bowel syndrome, inflammatory bowel disease, chronic diarrhea, constipation, colorectal polyps or cancer, a compromised immune system, metabolic syndromes, chronic fatigue syndrome, major GI surgery, or other diseases or conditions potentially associated with specific changes in fecal microbiota. Potential fecal microbiota donors are excluded if they exhibit positive laboratory tests for C-reactive protein, erythrocyte sedimentation rate, hepatitis A, hepatitis B, hepatitis C, human immunodeficiency virus, or syphilis. Potential fecal microbiota donors are excluded if they exhibit a positive test for stool ova or parasites. Potential fecal microbiota donors are excluded if they engage in high-risk sexual behaviors, have been incarcerated, or received any tattoos or body piercings in areas that have had disease epidemics within the past three months.

Donor fecal material (fresh feces) is collected in a sterilized container and then transferred to a blender. Approximately 500-1000 mL 0.9% saline solution is added to the blender and thoroughly mixed with the fecal sample. The resulting suspension is filtered at least 4 times through strainers prior to collecting a final suspension. The final suspension is centrifuged in 50 mL tubes at 1200×g for 3 minutes. The supernatant is discarded and the pellet is gently resuspended in approximately 50 mL of sterile 0.9% saline solution. The centrifugation and resuspension steps are repeated 2 to 4 additional times. Upon the final centrifugation, the supernatant is discarded. If the fecal microbiota is to be used immediately, the resultant pellet is resuspended in 1.5-volumes of 0.9% saline solution by gently mixing. If the fecal microbiota is to be stored, the resultant pellet is resuspended in 10% sterile glycerol and stored at −80 degrees Centigrade. If fecal microbiota is frozen, it is warmed to room temperature prior to administration to a patient.

### Example 2. Investigation of the Efficacy, Safety and Tolerability of Three-Day, Split-Dose Administration of Lyophilized Full-Spectrum Microbiota in Patients with Clostridium Difficile Infection (CDI)

A study is conducted to compare the efficacy of split-dose administration of Lyophilized Full-Spectrum Microbiota (L-FSM) in the treatment of patients with initial or recurrent episodes CDI. The primary safety objective is to evaluate the safety and tolerability of L-FSM in patients with initial or recurrent episodes of CDI over the 12 months following treatment. The secondary efficacy objectives are to evaluate the efficacy and safety of L-FSM in patients with initial or recurrent episodes of CDI associated with the ribosomal NAPI/BI/027 C. difficile subtype.

The study consists of a 7 day Screening Period, an 8-week Efficacy Assessment Period, a 16-week Safety Period, and a 26-week Safety Follow-Up Period. The total duration of study participation is 52 weeks. A total of five clinic visits and five telephone follow-up calls are conducted.

After providing written informed consent, patients will undergo a detailed medical history and physical examination with their history of CDI documented, including the current episode, along with results of culture, serologic testing, findings on colonoscopy, hospitalizations, complications, and treatments. Current and prior medications are reviewed, and clinical safety laboratory tests including a serum pregnancy test for females of childbearing potential (or follicle stimulating hormone [FSH] in women who are post-menopausal), and 12-lead electrocardiogram (ECG) are assessed. A stool sample is obtained for CDI culture, PCR toxin testing, and ribotyping including NAPI/BI/027 subtype, as well as parasites and enteric pathogen detection; a portion of this sample is stored for 16S ribosomal RNA testing/metagenomics DNA study. To be eligible for study participation, patients who have taken vancomycin complete 10 or more consecutive days of oral therapy at a dose of 125 mg QID or greater, with treatment being discontinued between 24-48 hours prior to L-FSM.

Multiple sets of dosing schedules are tested. In one set, eligible patients receive open-label L-FSM orally twice daily for 3 consecutive days; totaling 6 capsules per treatment. The first 30 patients receive high-dose L-FSM and the second 30 patients receive low-dose L-FSM (Table 1). Alternatively, different sets of dosing schedules are tested (Tables 2 and 3).

TABLE 1
Dosing schedule set 1.

| | Bacterial dose | Total Bacterial |
|---|---|---|
| Treatments: | per capsule | Dose |
| High-dose L-FSM | 1.0 × 10¹¹ | 6.0 × 10¹¹ |
| | organisms | organisms |
| Low-dose L-FSM | 1.0 × 10¹⁰ | 6.0 × 10¹⁰ |
| | organisms | organisms |

TABLE 2
Dosing schedule set 2.

| Treatment groups: | Total Bacterial Dose |
|---|---|
| Group 1: High-dose in capsule | 1.0 × 10¹¹ organisms |
| formulation in one day | |
| Group 2: High-dose in capsule | 3.0 × 10¹¹ organisms |
| formulation administered on three | |
| consecutive days | |
| Group 3: Low-dose in capsule | 1.0 × 10¹¹ organisms (amounting to |
| formulation administered on three | approximately 3.33 × 10¹⁰ per dose |
| consecutive days | on each day) |

TABLE 3
Dosing schedule set 3 using high-dose capsules.

| | | Total Bacterial |
|---|---|---|
| Treatments: | Dosing Regimen | Dose |
| Split-dose L-FSM | 2 capsules/day, | 6.0 × 10¹¹ |
| | 1 capsule each morning and | organisms |
| | evening, on Days 1 through 3 | |
| Single-dose L-FSM | 6 capsules as a single dose on | 6.0 × 10¹¹ |
| | Day 1 | organisms |

Patients return for clinic evaluations at Weeks 1, 4, and 8. At the discretion of the investigator, a home visit may be substituted for a telephone call. During clinic visits and telephone calls, patients are assessed for diarrhea, abdominal pain, fever, concomitant medications and adverse events. Stool samples are assessed for CDI by culture and polymerase chain reaction (PCR) at Weeks 1, 4 and 8. Fecal microbiome composition is evaluated for engraftment by 16S ribosomal RNA testing at all study visits. Clinical safety laboratory tests including urine pregnancy test for females of childbearing potential are performed at Weeks 1, 4, and 8.

Patients with untreated CDI, defined as persistence or recurrence of diarrhea (defined as >3 loose bowel movements (Bristol Stool Score 6 or 7) per day for at least 2 days) and positive stool for CDI by PCR or stool culture and at any time during the 8-week Efficacy Period are eligible to receive single open label treatment with high-dose CP101 or other therapy as deemed appropriate by the investigator. These patients are followed for an additional 48 weeks irrespective of response to investigational treatment. Patients with a positive stool for CDI by PCR or culture and potential ribotyping at Week 8 but who do not experience recurrence of diarrhea undergo same repeat stool testing at 12 and 16 weeks.

Patients are contacted by telephone at Weeks 12, 14, 36 and 48, at which time adverse events and concomitant medications are recorded. Patients reporting recurrence of diarrhea and positive stools for CDI after negative Week 4 or 8 evaluations are evaluated for re-infection by full re-testing of stool, including ribotyping if culture-positive, with comparison to screening samples.

Patients are discontinued from the study for any of the following:
1. Surgery or hospitalization, or need for a prohibited medication for treatment of CDI;
2. Adverse events that impact the safety of continued participation in this study;
3. Unwillingness to continue in this study;
4. Noncompliance with study drug, defined as use of less than 80% of scheduled drug doses, or any other aspect of the protocol;
5. Any other reason, based upon the medical judgment of the Investigator.

Patients who discontinue study participation before Week 8 are scheduled for an Early Termination Visit and undergo the procedures of Week 8 whenever possible.

The following are patient inclusion criteria of this study:
1. Ability to provide written informed consent from the patient, guardian, or caregiver;
2. Men or women 14 to 90 years of age;
3. Medical record documentation of CDI infection;
4. A positive stool test for CDI within 90 days of enrollment;
5. For patients with recurrent CDI, documented history of a prior response of an episode of CDI to antibiotics (Patients may alternately be eligible to participate if a colonoscopy in the past 12 months demonstrated evidence of inflammatory bowel disease, microscopic colitis, neoplasm, or other diarrhea-associated conditions);
6. Female patients of child bearing potential or male patients with a female partner/spouse of child bearing potential must be willing to use an established method of birth control during the study and be willing and able to continue contraception through the Week 24 visit. (Note: Women who are post-menopausal will have this confirmed by serum FSH.) Acceptable methods of contraception include barrier methods (condom, diaphragm, sponge), spermicide, oral contraceptives, depot contraceptives (implants/injectables) or IUD;
7. Ability to comply with study requirements.

The following are patient exclusion criteria of this study:
1. Women with a positive pregnancy test, who are breastfeeding, or who intend to become pregnant during the course of the study;
2. Toxic megacolon or ileus;
3. Presence of ileostomy or colostomy, or history of prior colon resection;
4. Existence of an abscess, enteric fistula, or symptomatic bowel obstruction;
5. Use of an antibiotic for any condition other than CDI at baseline or any anticipated use of an antibiotic for any condition other than CDI during the 8 week Efficacy Assessment Period;
6. Dysphagia or inability to swallow pills;
7. Anticipated use of antibiotics in the 6 months post enrollment;
8. Use of immunosuppressive agents, including but not limited to biologics, calcineurin inhibitors, thiopurines, methotrexate, and/or prednisone exceeding 10 mg/day, for any condition;
9. Prior fecal transplant for any condition within 12 months;
10. Tube feeding within 28 days of screening or planned during the course of treatment;
11. Stools positive for enteric infection, including parasitic, within 28 days of screening;
12. Active treatment for cancer (excluding non-melanomatous cancer of the skin or cervical carcinoma in situ) or lymphoproliferative disorders;
13. Active drug, chemical or alcohol dependency as determined by the Investigator;
14. Patients with planned hospitalization or surgery during the course of the study;
15. Life expectancy less than 48 weeks;
16. Participation in any other investigational study, or use of any other investigational medication, within 30 days of screening or at any time during the study;
17. Clinically significant disease: renal, hepatic, neurological, cardiovascular, pulmonary, endocrinologic, psychiatric, hematologic, urologic, or other acute or chronic illness that in the opinion of the Investigator would not make the patient a suitable candidate for this study; or other unspecified reasons that, in the opinion of the Investigator or the Sponsor, make the patient unsuitable for enrollment.

### Example 3. Treatment of First-Time Clostridium Difficile Infection with Fecal Microbiota Transplantation

A study is performed to assess the effectiveness of Fecal Microbiota Transplantation (FMT) as a first line treatment for patients with initial diagnosis of Clostridium difficile infection (CDI). Previously, CDI is treated with FMT after two failed treatments with antibiotics, e.g., vancomycin or metronidazole. See Cammarota et al., Aliment Phrmacol. Ther., 41:835-43(2015). Success rate of FMT in relapsing CDI is reported to be close to 90%. However, because CDI often follows antibiotic use which may result in depleted bacterial classes, FMT treatment is pursued to eradicate CDI and replace missing flora components simultaneously rather than another antibiotic treatment. FMT is provided to patients with initial diagnosis of CDI.

Consecutive patients are selected with toxin-positive CDI (toxin assay or PCR) upon stool test analysis. Patients with inflammatory bowel diseases are excluded in this study. Two FMT infusions are given; the initial via colonoscopy, followed next day by rectal enema. Patients report minor adverse effects resembling Irritable Bowel Syndrome (IBS) symptoms including; bloating, abdominal pain, excessive flatulence and nausea. Symptom data are recorded at initial presentation prior to FMT, and again at 8 weeks and at 26 weeks up to 2 years post FMT.

To maintain uniform approach, pre-FMT antibiotic treatment is provided. For pre-treatment, fifteen patients are given vancomycin 500 mg twice daily (BD), six are given vancomycin 500 mg BD and Rifaximin 1000 mg BD, four are given vancomycin 500 mg BD and metronidazole 200 mg BD, two are given vancomycin 250 mg mane and 500 mg nocte, one is given 500 mg Rifaximin daily, and the final patient is given no pre-treatment. Stool cultures and toxin assays are repeated after 4 weeks. Patients are seen in clinic approximately 8 weeks after FMT as part of standard-of-care follow-up. Treatment success is defined as negative stool culture and toxin assay.

All 29 patients recruited are cured of CDI achieving 100% eradication. Seven patients (26%) report transient bloating and flatulence immediately after FMT. Ten patients (34%) report a decline in bowel motions from an average of 5.9 BM to 1.6 BM per day. Five patients (17%) report constipation initially. Overall, the lessening of initial symptom severity is achieved in fifteen patients (55%).

In conclusion, in patients with initial diagnosis of CDI, FMT is observed as a safe and 100% effective treatment of CDI. Transient adverse effects of bloating/flatulence are noted. Accompanying IBS can persist after CDI cure in this cohort albeit moderate improvement occurs in some patients.

### Example 4. Optimal Storage Conditions of Full-Spectrum Microbiota (FSM) for Fecal Microbiota Transplantation

A study is performed to assess the number of viable cells associated with alternate storage formats of FMT. Briefly, fresh stool from a healthy donor is collected within 1 hour of bowel motion. Homogenization of the stool is performed with sucrose, trehalose, and saline. The resulting slurry is filtered with a 250 micron filter bag. The fresh FSM samples are divided into four groups. Samples in Group 1 are taken at this point for bacterial viability testing. Samples in Groups 2 and 3 are stored for 2 days at −20° C. and −80° C., respectively. The remaining FSM liquid (Group 4) is centrifuged then lyophilized (freeze-dried) before viability testing. Bacterial viability is determined using Live/Dead® BacLight™ Bacterial Viability staining kit (ThermoFisher Scientific, Waltham, Mass.) via flow cytometry.

Fresh FSM has the highest percentage of live cells at 69.5%, followed by lyophilized FSM at 55.1% and frozen FSM stored at −20° C. with 40.4% (See Table 4). Frozen FSM stored at −80° C. has the lowest percentage of live cells with 36.2%. These data indicate that FSM fecal microbiota remains viable regardless of storage conditions and format, albeit at varying concentrations. Results demonstrate lyophilized FSM as an alternative to conventional liquid FSM.

TABLE 4
Bacterial cell viability under various storage conditions

| | | Live | Total | |
|---|---|---|---|---|
| Format | Replicate | Cells/mL | Cells/mL | % Live Cells |
| Fresh FSM | A | 8.64E+09 | 1.30E+10 | 66.6 |
| | B | 9.89E+09 | 1.34E+10 | 73.8 |
| | C | 1.04E+10 | 1.52E+10 | 68.1 |
| | Average | 9.63E+09 | 1.39E+10 | 69.5 |
| | | | | (SEM = 2.19) |
| Frozen FSM | A | 1.75E+10 | 3.33E+10 | 52.5 |
| (stored at −20° C. | B | 8.71E+09 | 2.54E+10 | 34.3 |
| for 2 | C | 1.16E+10 | 3.37E+10 | 34.5 |
| days) | Average | 1.3E+10 | 3.1E+10 | 40.4 |
| | | | | (SEM = 6.03) |
| Frozen FSM | A | 6.20E+09 | 1.81E+10 | 34.2 |
| (stored at −80° C. | B | 5.90E+09 | 1.91E+10 | 30.9 |
| for 2 | C | 1.07E+10 | 2.46E+10 | 43.3 |
| days) | Average | 7.6E+09 | 2.1E+10 | 36.2 |
| | | | | (SEM = 3.71) |
| Lyophilized | A | 4.10E+11 | 7.80E+11 | 52.6 |
| Reconstituted | B | 3.07E+11 | 6.66E+11 | 46.1 |
| FSM* | C | 4.54E+11 | 6.83E+11 | 66.6 |
| | Average | 3.9E+11 | 7.1E+11 | 55.096 |
| | | | | (SEM = 6.05) |

### Example 5. Encapsulated Lyophilized Full-Spectrum Microbiota for Treatment of Initial and Recurrent Clostridium difficile Infection

A study is performed to assess the safety of encapsulated lyophilized FSM (L-FSM) in CDI. Briefly, stool donors are screened following AGA guidelines. The stool is processed within 6 hours of motion; homogenized, filtered then concentrated with a combination of saline and cryoprotectant (sucrose and trehalose). The resulting slurry is then filtered, centrifuged, lyophilized and encapsulated. Capsules are stored at −80° C. and when dispensed are kept by patients at 4° C. Relapsing CDI patients maintain their antibiotics until two days prior to L-FSM capsule use, then cease and patients with initial CDI are pre-treated with vancomycin to reduce colonization resistance. Patients ingest 6 or 8 capsules over one or three days. Patient stool samples are submitted for CDI analysis (PCR and culture) at 1, 2, 4, 6, and 12 weeks post-treatment. Patients maintain a symptom diary throughout the course of treatment.

As shown in Table 5, poor patient stool collection results in incomplete data. The relocation of patients 2 and 3 results in incomplete data for weeks 6 and 12. Minimal adverse events are reported including diarrhea, constipation, flatulence, abdominal discomfort, nausea and bloating. In 6 of 7 treated patients, C. difficile is not present at or beyond week 4 post-treatment. Further, positive toxin gene PCR in stool may be detectable for 1-2 weeks post treatment. Overall encapsulated L-FSM eradicated CDI in 85% of patients, in this small patient case series. Lyophilized encapsulated FMT is safe and well tolerated with no significant adverse events reported. This study demonstrates that encapsulated L-FSM product—can replace current liquid stool CDI treatment.

TABLE 5
Lyophilized FSM Capsule Use in Initial and Recurrent Clostridium difficile Infection

| Patient | Antibiotic | Antibiotic | CDI | Capsule | Week Post FSM Capsule Use | | | | |
|---|---|---|---|---|---|---|---|---|---|
| Number | Failure | Pre-treatment | Status | Number | 1 | 2 | 4 | 6 | 12 |
| 1 | No | Yes | Initial | 8 | − | − | − | \ | \ |
| 2 | Yes | Yes | Recurrent | 8 | + | − | − | N/A^{#} | N/A^{#} |
| 3 | Yes | No | 2nd | 8 | \ | \ | − | N/A^{#} | N/A^{#} |
| 4 | No | Yes | Initial | 6 | − | − | − | − | − |
| 5 | Yes | Yes | 2nd | 6 | − | − | − | − | − |
| 6 | No | Yes | Initial | 6 | − | \ | \ | − | − |
| 7 | No | Yes | Initial | 6 | − | − | + | + | + |

^{#}Patients are unable to be contacted.
A diagonal stroke indicates lack of stool specimen submitted.

### Example 6. Patient with Relapsing C. difficile Successfully Treated with Encapsulated Lyophilized Full-Spectrum Microbiota

A 29 year-old male with relapsing PCR positive CDI is recruited after treatment failure with vancomycin. In preparation for the patient's treatment, a stool donor is subjected to stool and serological tests as per AGA Consensus Guidance on Donor Screening and Stool Testing for FMT. Collected stool is processed within 6 hours of being passed—homogenized, filtered and concentrated with a combination of saline and cryoprotectant (sucrose and trehalose). Resulting slurry is lyophilized and encapsulated in gel capsules (Capsugel delayed release veggie caps size 00) resulting in 1.57×10¹¹ viable cells per capsule. Capsules are subsequently stored at −80° C.

The capsules are ingested by the patient 2 days after a final dose of nine days of vancomycin. The patient ingests two L-FSM capsules in the morning and evening with food over two days, totaling eight capsules. The patient submits stool samples for testing on Days 10, 25 and 34, post treatment to confirm C. difficile eradication. Further, questionnaires detailing symptoms and adverse events are completed prior to and 10 days post treatment with patient follow up continuing for an additional seven weeks.

Stool PCR and culture on Days 10, 25 and 34, post capsule treatment are negative for C. difficile. The patient reports a significant decrease in bowel motions from great than 9/day prior to treatment to 2/day following treatment. The patient also reports a decrease in frequency of bloating from ‘always’ to ‘rarely’ following L-FSM, and an improvement in the initially moderate-severe abdominal pain. Additional patient follow up is lost due to relocation to another country.

This case study outlines the successful treatment of using encapsulated lyophilized FSM in a patient with CDI previously treated unsuccessfully with vancomycin.

### Example 7. A Multi-Day Dosing Regimen Leads to a Higher Remission Rate Compared to a Single-Day Dosing Regimen for Treating C. difficile Infection (CDI) Using L-FSM Capsules

Eighteen patients with an initial or recurrent CDI are recruited and treated with Lyophilized Full-Spectrum Microbiota (L-FSM). Patients are treated via a protocol essentially following the summary in Example 2. L-FSM capsules are manufactured essentially as in Example 5. However, the only deviation in this study has been the addition of sucrose to phosphate buffered saline rather than using saline, trehalose and sucrose combination. Two dosing regimens, similar to the dosing schedule set 3 in Table 3, are employed: a single-day dosing and a multi-day dosing. Details of the two regimens are listed in Table 6. A multi-day regimen comprises the ingestion of a total of 6 or 8 capsules across 2 to 3 days. For example, patients 1 to 3 take two capsules each time, twice a day, for two consecutive days (indicated as “2×2 over 2 days”). Patients 6 to 9, 11, and 12 take one capsule each time, twice a day, for three consecutive days (indicated as “1×2 over 3 days”), while patient 10 takes one capsule each time, four times a day, for two consecutive days (indicated as “1×4 over 2 days”). A single-day regimen comprises the ingestion of 6 capsules within one day. For example, patients 13 to 17 take 6 capsules at once (indicated as “6×1 over 1 day”).

Patient symptoms are evaluated around 8 weeks after the L-FSM treatment. Patients are considered to have achieved remission or cure if they are diarrhea-free and C. difficile-negative by PCR test on patient's stool. Relapse of CDI is defined as diarrhea with C. difficile-positive stool by PCR by Week 8.

For the six patients that receive a single-day regimen, four of them achieve remission. The remission rate from the single-day regimen is about 66.7% (4 out 6) for this patient group. For patients that receive a multi-day regimen, 11 out of 12 achieve remission (including patient 18b, but not patient 5 given the lack of colon and the much higher number of capsules taken). This translates into a remission rate of about 91.7% for the multi-day regimen. If multi-day regimen patients are limited to those that only take the same number of capsules (i.e., 6 capsules) as by the single-day regimen patients, 6 out of 7 multi-day regimen patients achieve remission. This points to a remission rate of about 85.7%. Therefore, in this group of CDI patients, a multi-day regimen provides a higher remission rate compared to a single-day regimen when treated with L-FSM capsules.

As various modifications could be made in the constructions and methods herein described and illustrated without departing from the scope of the disclosure, it is intended that the foregoing description shall be interpreted as illustrative rather than limiting. The breadth and scope of the present disclosure should not be limited by any of the above-described exemplary embodiments, but should be defined only in accordance with the following claims appended hereto and their equivalents. All patent and non-patent documents cited in this specification are incorporated herein by reference in their entireties.

TABLE 6
Patient data showing a single-day dosing regimen compared multi-day dosing regimen
for the treatment of CDI by L-FSM capsules.

| | Regimens | | | | | |
|---|---|---|---|---|---|---|
| | (“capsules per intake” × “number of | | Total | | | |
| | intake per day” over “number of days”) | | Number | | | |
| Patient | Single-day | | of | Live cells | Total cells | |
| No. | dosing | Multi-day dosing | Capsules | per gram | per gram | Remission |
| 1 | | 2 × 2 over 2 days | 8 | 1.25E+12 | 1.28E+12 | Yes |
| 2 | | 2 × 2 over 2 days | 8 | 6.19E+11 | 7.80E+11 | Yes |
| 3 | | 2 × 2 over 2 days | 8 | 1.11E+11 | 1.18E+11 | Yes |
| 4 | | 1 × 2 over 3 days | 6 | 1.40E+11 | 1.49E+11 | Yes |
| 5* | | Multiple (5 weeks) | 92 | Average | Average | Yes |
| | | | | 8.21E+10 | 1.08E+11 | |
| 6 | | 1 × 2 over 3 days | 6 | 1.33E+11 | 1.85E+11 | No; |
| | | | | | | relapsed** |
| 7 | | 1 × 2 over 3 days | 6 | 1.59E+11 | 2.05E+11 | Yes |
| 8 | | 1 × 2 over 3 days | 6 | 2.49E+11 | 3.05E+11 | Yes |
| 9 | | 1 × 2 over 3 days | 6 | 5.28E+10 | 2.79E+11 | Yes |
| 10 | | 1 × 4 over 2 days | 8 | 6.36E+10 | 2.88E+11 | Yes |
| 11 | | 1 × 2 over 3 days | 6 | N/A | N/A | Yes |
| 12 | | 1 × 2 over 3 days | 6 | N/A | N/A | Yes |
| 13 | 6 × 1 over 1 day | | 6 | 1.54E+11 | 2.07E+11 | No; relapsed |
| 14 | 6 × 1 over 1 day | | 6 | 1.20E+11 | 2.41E+11 | Yes |
| 15 | 6 × 1 over 1 day | | 6 | 5.61E+10 | 8.93E+10 | Yes |
| 16 | 3 × 2 over 1 day | | 6 | N/A | N/A | Yes |
| 17 | 6 × 1 over 1 day | | 6 | N/A | N/A | Yes |
| 18a*** | 3 × 2 over 1 day | | 6 | N/A | N/A | No; relapsed |
| 18b | | 2 × 3 over 2 days | 12 | N/A | N/A | Yes |

*Patient has C. diff infection in small bowel (patient's colon resected for polyps).
**Patient experiences a relapse and is subsequently treated with enema-based FMT.
***Patient first experiences a relapse after a first treatment based on a single-day regimen (18a) and subsequently achieves remission after switching to a multi-day regimen (18b).
N/A: The live and total cell counts are not available for the L-FSM materials used to make the capsules given to these patients.

1.-28. (canceled)

29. A method for treating a gastrointestinal disorder in a subject in need thereof, said method comprising administering to said subject a pharmaceutically active dose of a composition comprising live non-pathogenic fecal bacteria, said dose being administered at a first dosing schedule of 2 or more days, wherein said first dosing schedule is capable of achieving an increase in the remission or cure rate of said gastrointestinal disorder compared to a second dosing schedule where a similar total amount of said composition is administered in a single day.

30. The method of claim 29, wherein said increase is at least about 10%.

31. The method of claim 29, wherein said first dosing schedule achieves a remission or cure rate of at least 80%.

32. The method of claim 29, wherein said gastrointestinal disorder is a Clostridium difficile infection (CDI).

33. The method of claim 32, wherein said CDI is a recurrent CDI.

34. The method of claim 29, wherein said first dosing schedule comprises administering said dose once daily for 3 days.

35. The method of claim 34, wherein said 3 days are 3 consecutive days.

36. The method of claim 29, wherein said composition comprises a donor's substantially complete microbiota.

37. The method of claim 29, wherein said composition comprises a fecal microbe preparation produced from a donor stool via ethanol treatment.

38. The method of claim 29, wherein said composition is frozen.

39. The method of claim 29, wherein said administering is administering orally.

40. The method of claim 29, wherein said pharmaceutically active dose comprises at least about 10⁸ total cell count.

41. The method of claim 29, wherein said pharmaceutically active dose comprises at most about 10⁹ total cell count.

42. The method of claim 29, wherein said pharmaceutically active dose comprises from 10⁸ to 10¹⁴ total cell count.

43. The method of claim 29, wherein said non-pathogenic fecal bacteria are spores.

44. A method for treating a recurrent Clostridium difficile infection (CDI) in a subject in need thereof, said method comprising administering to said subject a pharmaceutically active dose of a composition comprising non-pathogenic fecal bacterial spores, said dose being administered for three days, wherein said pharmaceutically active dose comprises from 10⁸ to 10¹⁴ total cell count.

45. The method of claim 44, wherein said composition comprises a fecal microbe preparation produced from a donor stool via ethanol treatment.

46. A method for treating a Clostridium difficile infection (CDI) in a subject in need thereof, said method comprising orally administering to said subject a pharmaceutically active dose of a composition comprising non-pathogenic fecal bacteria, said dose being administered for three consecutive days, and wherein said composition comprises a fecal microbe preparation produced from a donor stool via ethanol treatment.

47. The method of claim 46, wherein said CDI is a recurrent CDI, and wherein said dose is administered once daily.

48. The method of claim 46, wherein said subject is pretreated with an antibiotic and/or proton pump inhibitor prior to administration of said composition.
